(19) United States
(12) Patent Application Publication
(10) Pub. No.: US 2019/0300271 A1
(43) Pub. Date: Oct. 3, 2019

(54) DISPENSER APPARATUS AND METHOD FOR DISPENSING DENTAL X-RAY BARRIER ENVELOPES

(71) Applicants: Frederick DESANTI, Huntington Station, NY (US); Michael DESANTI, Huntington Station, NY (US); Howard S. GLAZER, Paramus, NJ (US)

(72) Inventors: Frederick DESANTI, Huntington Station, NY (US); Michael DESANTI, Huntington Station, NY (US); Howard S. GLAZER, Paramus, NJ (US)

(21) Appl. No.: 16/368,143

(22) Filed: Mar. 28, 2019

Related U.S. Application Data
(60) Provisional application No. 62/650,357, filed on Mar. 30, 2018.

(51) Int. Cl. B65D 83/08; B65D 85/672

(52) U.S. Cl. CPC B65D 83/0805; A61B 6/4423; B65D 85/672; B65D 83/0894

(57) ABSTRACT

A system for dispensing X-ray barrier envelopes comprises an elongated flexible substrate having a front side and a rear side, and a plurality of X-ray barrier envelopes removably disposed on the substrate and arranged along the front side of the substrate. The substrate with the X-ray barrier envelopes is configured to be wound around a spool so that the envelopes can be dispensed by sequentially removing each envelope from the end of the spooled substrate. As the envelopes are removed from the substrate, the substrate can be trimmed or torn off so that an envelope is always disposed at the end of the substrate.

### CROSS-REFERENCE TO RELATED APPLICATIONS

Applicant claims priority under 35 U.S.C. § 119(e) of U.S. Provisional Patent Application Ser. No. 62/650,357, filed on Mar. 30, 2018, the disclosure of which is incorporated by reference.

### BACKGROUND OF THE INVENTION

### 1. Field of the Invention

This invention relates to a dispenser apparatus for dispensing X-ray barrier envelopes, and to method for dispensing the envelopes.

### 2. The Prior Art

Dental X-ray barrier envelopes along with phosphor sensor X-ray plates are used extensively worldwide in the field of dentistry. When dental X-rays are taken, the phosphor plates are placed by hand inside of a disposable X-ray barrier envelope and then placed inside the patient's mouth. After the X-rays are taken the sensor plates are removed by hand from the barrier envelope, processed and the disposable X-ray barrier envelope is discarded. The amount of X-rays necessary will vary from patient to patient, which determines how many times this procedure is repeated.

By design, these small barrier envelopes are made of an ultra-thin soft material, making them cumbersome and difficult to handle when opening and inserting the phosphor X-ray plates. This procedure becomes even more difficult while wearing protective gloves. It would be desirable to develop a system that allows for easier placement of the phosphor plates into the barrier envelopes.

### SUMMARY OF THE INVENTION

It is therefore an object of the invention to provide a system that allows for the barrier envelopes to be dispensed more quickly and easily.

This object is accomplished by a dispensing system comprising an elongated flexible substrate having a front side and a rear side, and a plurality of X-ray barrier envelopes removably disposed on the substrate and arranged along the front side of the substrate. The substrate with the X-ray barrier envelopes is configured to be wound around a spool so that the envelopes can be dispensed by sequentially removing each envelope from the end of the spooled substrate. As the envelopes are removed from the substrate, the substrate can be trimmed or torn off so that an envelope is always disposed at the end of the substrate.

In one embodiment, the X-ray barrier envelopes are arranged so that each envelope partially overlaps an adjacent envelope. In another embodiment, the barrier envelopes are arranged so that each envelope is arranged so as to not contact an adjacent envelope.

In a preferred embodiment, the spooled substrate is arranged on an axle that extends through the spool, and the spool and axle are disposed in a storage box. The axle is rotatably mounted to the storage box so that the coil can be unwound by pulling the end of the substrate. The end of the substrate extends through a slot in the storage box for simple dispensing. The row of X-ray barrier envelopes may be marked or coded so that the user can easily determine how many barrier envelopes remain on the spool. In another embodiment, multiple spools of substrate can be arranged on the axle, to dispense X-ray barrier envelopes of different sizes. Alternatively, the axle could be mounted on a wall bracket and the spool could be open to access by the user.

In a preferred embodiment, the X-ray barrier envelopes are mounted to the substrate using a releasable adhesive. Alternatively, each envelope can be disposed inside a pocket in the substrate.

The substrate is preferably formed from a paper material, such as corrugated cardboard. Other materials, such as plastic could also be used.

In another embodiment, no substrate is used, and each barrier envelope is connected to an adjacent barrier envelope and rolled into a spool. The envelopes are separable from each other by perforations or other weakening configuration of the envelope material.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a spool of substrate according to the invention;

FIG. 2 shows an example of X-ray barrier envelopes used in the system of the invention;

FIG. 3 shows the envelopes of FIG. 2 containing phosphor plates;

FIG. 4 shows the spooled substrate mounted in a dispensing container; and

FIG. 5 shows a spool of the envelopes without a substrate.

### DETAILED DESCRIPTION OF THE EMBODIMENTS

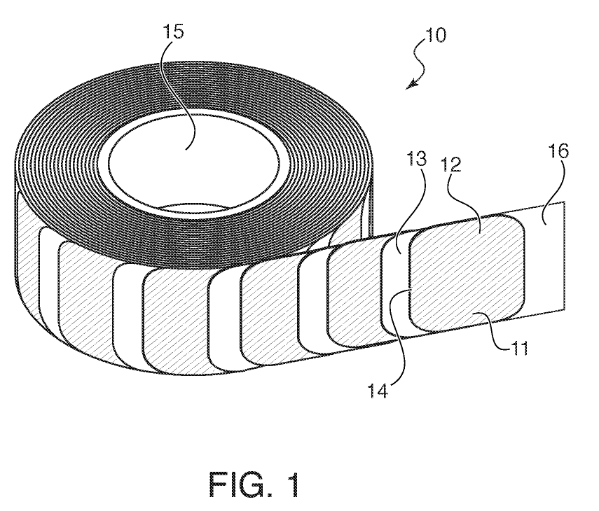

Referring now in detail to the drawings, FIG. 1 shows the system 10, which comprises a substrate 16 rolled on a spool 15. Substrate 16 contains a plurality of X-ray barrier envelopes 11, which are arranged overlapping one another along substrate 16 throughout the length of substrate 16. Each envelope 11 is attached to substrate 16 via a non-toxic removable adhesive, which allows the envelopes 11 to be easily peeled off of the substrate. Alternatively, each envelope could be disposed in a pocket created in the substrate. The substrate 16 could be made of any suitable material, such as corrugated paper or plastic tape.

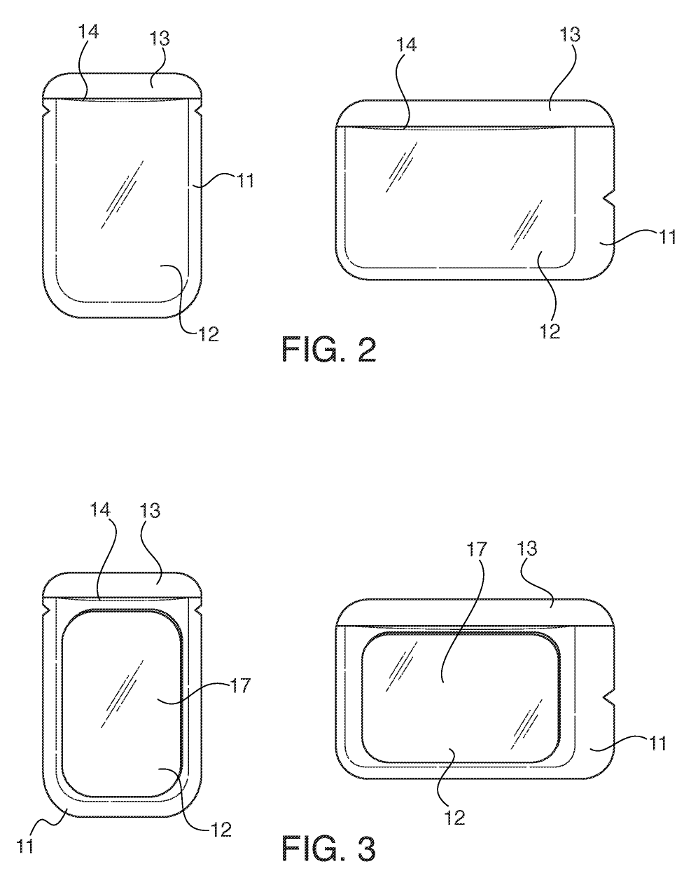

FIGS. 2 and 3 show barrier envelopes 11 in two separate orientations. Barrier envelopes 11 have a front 12, a rear 13 and an opening 14 for insertion of a phosphor plate 17, as shown in FIG. 3. In use, each envelope 11 is peeled off or removed from the spool of substrate 16 and a phosphor plate 17 is inserted therein prior to use on a patient. The present invention makes the assembly of the envelope 11 and plate 17 much quicker and simpler, as it keeps each envelope separated from the others for individual assembly.

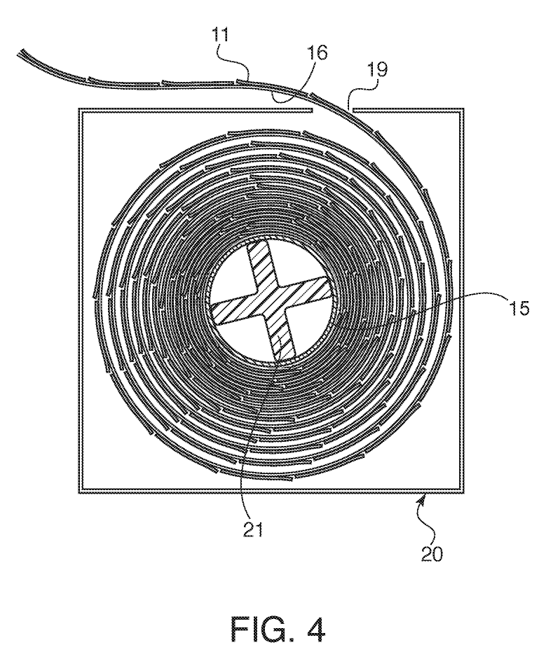

FIG. 4 shows the spool of substrate 16 with envelopes 11 mounted in a dispensing container 20. An axle 21 is placed inside spool 15 and is mounted to the sides of container 20 so as to be rotatable. The end of substrate 16 extends through a slot 19 in the top of container 20 to allow substrate 16 to be unspooled by pulling on the end. Alternatively, the axle could be mounted to a bracket and hung on a wall, without the container 20. In another embodiment, several spools of substrate 16 can be mounted side-by-side on a single axle for dispensing, either with or without container 20.

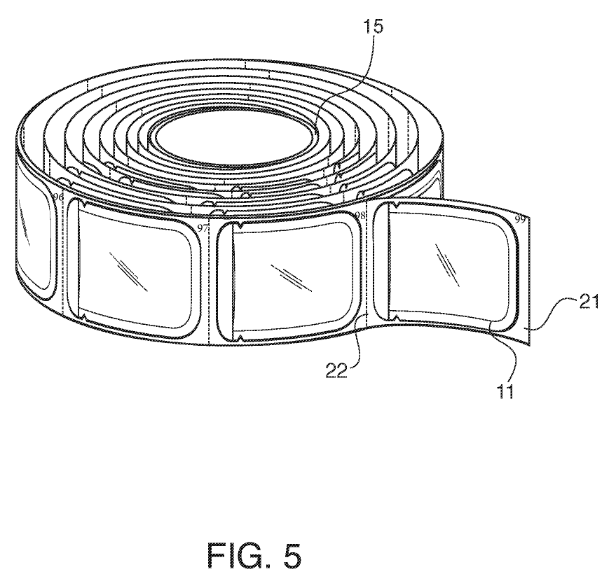

FIG. 5 shows yet another alternative embodiment, in which envelopes 11 are not mounted on a substrate, but have an extended border 21 connected to each other by a perforation 22. The connected envelopes 11 are rolled directly around spool 15. Each envelope 11 is torn or cut off from the previous envelope along perforation 22, with no need to dispose of a substrate.

The envelopes 11 in any of the embodiments can be labeled to indicate how many envelopes are left on the spool, such as by numbers as shown or with other indicia.

Accordingly, while only a few embodiments of the present invention have been shown and described, it is obvious that many changes and modifications may be made thereunto without departing from the spirit and scope of the invention.

What is claimed is:

1. A system for dispensing X-ray barrier envelopes, comprising:
an elongated flexible substrate having a front side and a rear side, and
a plurality of X-ray barrier envelopes removably disposed on the substrate and arranged along the front side of the substrate.

2. The system according to claim 1, wherein the X-ray barrier envelopes are arranged so that each envelope partially overlaps an adjacent envelope.

3. The system according to claim 1, wherein the substrate is wound on a spool.

4. The system according to claim 1, further comprising:
an axle extending through the spool, wherein the axle is configured to be rotatably mounted so that substrate can be unwound by pulling on an end of the spool.

5. The system according to claim 5, wherein the spool is disposed in a storage box having a slot, wherein the end of the substrate extends through the slot so that the substrate can be unwound from the spool by pulling the end of the substrate.

6. The system according to claim 5, wherein the axle is rotatably mounted to sides of the storage box

7. The system according to claim 4, wherein there are multiple spools of substrate arranged on the axle.

8. The system according to claim 1, wherein the X-ray barrier envelopes are mounted to the substrate using a releasable adhesive.

9. The system according to claim 1, wherein the substrate is formed from paper or plastic.

10. A system for dispensing X-ray barrier envelopes comprising a plurality of X-ray barrier envelopes arranged in a single row, each barrier envelope being connected to an adjacent barrier envelope and being separable from the adjacent barrier envelope.

11. The system according to claim 9, wherein the row of envelopes is wound on a spool.
